(19) United States
(12) Patent Application Publication
(10) Pub. No.: US 2007/0158455 A1
(43) Pub. Date: Jul. 12, 2007

(54) Disposable pierced earring fragrance chamber

(76) Inventor: Kathryn S. Robbins-Sullivan, San Marino, CA (US)

Correspondence Address: ALBERT O COTA, 5460 WHITE OAK AVE, SUITE A-331, ENCINO, CA, 91316, US

(21) Appl. No.: 11/328,598

(22) Filed: Jan. 11, 2006

(51) Int. Cl. A24F 25/00; A61L 9/04

(52) U.S. Cl. 239/34; 239/41; 239/42; 239/43

(57) ABSTRACT

A disposable fragrance chamber (10) for dispensing a fragrance from an aromatic liquid. The disposable fragrance chamber (10) consists of an outer casing body (20) having pad exposure openings (24), a detachable lid (34) and an absorbent pad (30) disposed between the body (20) and the lid (34). The chamber (10) permits an aromatic liquid to be applied into the absorbent pad (30) through the pad exposure openings (24). The aromatic liquid emits a fragrance into the surrounding environment when the chamber (10) is installed on an earring post (40) that extends beyond a person's ear lobe (48) held in place with an earring clasp (50).

### TECHNICAL FIELD

The invention generally pertains to fragrance dispensers, and more specifically to a disposable fragrance chamber that is worn by a person between a pierced earring and a earring clasp.

### BACKGROUND ART

Currently, there are many types of jewelry dispensers for perfumes and medications that are available to provide a means for dispensing the fragrance of perfume over a period of time.

The prior art listed below did not disclose patents that possess any of the novelty of the instant invention; however the following U.S. patents are considered related:

| U.S. Pat. No. | Inventor | Issue Date |
|---|---|---|
| 4,159,631 | Lee | Jul. 3, 1979 |
| 4,353,370 | Evans | Oct. 12, 1982 |
| 4,452,052 | Hodge | Jun. 5, 1984 |
| 5,031,419 | Gleman | Jul. 16, 1981 |
| 6,662,596 B2 | Tsutsumi | Dec. 16, 2003 |

Lee in U.S. Pat. No. 4,159,631 teaches a perfumed vapor dispensing article of jewelry in the shape of a hollow sphere having upper and lower halves secured together. A disc having gauze-filled cutouts is supported within the housing and wicks extend between sections of the gauze. Orifices in the upper half permit the perfumed vapors to escape. A hypodermic needle is inserted into the sphere's lower half to supply perfume.

In another embodiment the rod extends through the central aperture with removal of the rod permitting the lower half to be filled with an eyedropper.

U.S. Pat. No. 4,353,370 issued to Evans is for a medicated ear rod having a groove containing a cleaning agent and a stopper member utilizing a cotton saturated with alcohol or hydrogen peroxide that grasps the rod.

Hodge in U.S. Pat. No. 4,452,052 discloses an earring structure having an interior compartment which houses a removable perfume cartridge. The cartridge has a base containing the perfume, with a rotatable top having an aperture through which fragrance may escape. Rotation of the top piece changes the aperture area, thereby permitting the wearer to block the escape of fragrance.

Gleman in U.S. Pat. No. 5,031,419 teaches an earring clasp for pierced earrings which disposes a perfumed agent adjacent to or in contact with the person's ear lobe. The clasp is formed having a first and second disc member, and a body of sorbet material sandwiched in between the discs. A plurality of air holes permit air flow and in one embodiment the first disc is disposed adjacent to the person's ear lobe and is formed of a fluid pervious material to permit capillary transmission of the perfuming agent.

U.S. Pat. No. 6,662,596 B2 issued to Tsutsumi teaches a fragrance diffuser which covers the tip of the earring post to eliminate injury to the wearer. An outer case is mounted over the catch having an absorbent member with perfume impregnated therein. A number of openings in the outer case permit the fragrance to escape and a filling port permits the perfume such as the pipe from a spray nozzle to be inserted to impregnate the absorbent material.

For background purposes and as indicative of the art to which the invention is related reference may be made to the remaining cited patents issued to Wachter in U.S. Pat. No. 152,197.

### DISCLOSURE OF THE INVENTION

Previously, aromatic liquids such as perfume have been applied directly to a person's skin in a convenient area, such as behind the ears or on the wrist, in order to produce a pleasant fragrance for a period of time. While this method of application is easy to perform and achieves the purpose, the fragrance is often short-lived as its intensity decreases with wearing and is eliminated by bathing. Additionally, some persons are allergic or at least sensitive to an aromatic liquid when it is applied directly to the skin.

Therefore, the primary object of the invention is to keep aromatic liquids from directly touching a person's skin. Application of the aromatic is accomplished by using a dropper to directly apply the aromatic liquid into a set of four openings located on one side of a fragrance chamber. When it is worn, the fragrance chamber faces away from the skin, and the aromatic liquid is absorbed into the chamber, thus releasing a fragrance from the four openings.

In addition to the primary object of the invention, it is also an object of the invention to produce a disposable pierced earring fragrance chamber that:
is more sanitary then existing fragrance devices that are refillable and that require periodic cleaning,
is disposable and the aromatic liquid can be changed without refilling, thus aromatic liquids are not mixed with each other,
utilizes a chamber that is hypo-allergenic since it is made of a thermoplastic that has been approved to be in direct contact with the human body,
provides a fragrance chamber that is easily handled,
when an aromatic liquid is applied into the chamber the fragrance lasts longer,
by using the chamber, an aromatic liquid does not change on the wearer, as it does when placed directly on the skin.
when mounted onto an earring post, the post is completely protected from the aromatic liquid because of an interlocking design feature within the center of the chamber that the post slides in and out of,
eliminates the chance of an aromatic liquid getting into the pierced opening of the wearer. This isolation of the aromatic liquid form the earring post is important since the post can become contaminated with the aromatic liquid.
can be used with almost any aromatic liquid including perfume, essential oils or insect repellents. This is important as the aromatic liquid never touches a person's skin, therefore any type of aromatic liquid may be used with complete confidence,
utilizes a pad within the fragrance chamber that is flesh colored, and the outer casing body of the chamber is translucent with a transparent inner casing lid such that the flesh colored material of the fragrance chamber is visually obscured, thus causing the invention to be unobtrusive and to visually blend with the wearer's skin.
is so lightweight that it is hardly felt by the wearer,
the chamber may be used with any type of normal pierced earring posts,
can also be furnished in a kit form with a small eye dropper, extra disposable chambers, aromatic liquids and the like,
unlike other fragrance diffusers, the chamber has a large flat surface that supports the ear lobe in a vertical position, when wearing large earrings, and
is cost effective since it is made by injection molding. Once the tooling cost is amortized the individual piece price is minimal when produced in quantity due to the economy of numbers. The pads are likewise inexpensive to produce using multiple unit steel rule dies and conventional punch presses, thus inexpensive for the public to buy.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is a partial isometric view of a person's ear with the fragrance chamber on the back of the ear lobe retained by the pierced earring post and held in place with a earring clasp in the preferred embodiment.

FIG. 2 is a partial isometric view of the fragrance chamber in the preferred embodiment.

FIG. 3 is a cross-sectional view taken along lines 3-3 of FIG. 2.

FIG. 4 is a partial isometric view of the inner casing lid of the preferred embodiment shown removed from the invention for clarity.

FIG. 5 is a rear view of the inner casing lid of the preferred embodiment shown removed from the invention for clarity.

FIG. 6 is a cross-sectional view taken along lines 6-6 of FIG. 5.

FIG. 7 is a front view of the inner casing lid of the preferred embodiment shown removed from the invention for clarity.

FIG. 8 is a partial isometric view of the outer casing body of the preferred embodiment shown removed from the invention for clarity.

FIG. 9 is a front view of the outer casing body of the preferred embodiment shown removed from the invention for clarity.

FIG. 10 is a cross-sectional view taken along lines 10-10 of FIG. 9.

FIG. 11 is a rear view of the outer casing body of the preferred embodiment shown removed from the invention for clarity.

FIG. 12 is a partial isometric view of the absorbent pad of the preferred embodiment shown removed from the invention for clarity.

FIG. 13 is a rear view of the absorbent pad of the preferred embodiment shown removed from the invention for clarity.

FIG. 14 is a cross-sectional view taken along lines 14-14 of FIG. 13.

FIG. 15 is a front view of the absorbent pad of the preferred embodiment shown removed from the invention for clarity.

FIG. 16 is an exploded view of the pierced earring fragrance chamber in the preferred embodiment including an earring and clasp.

FIG. 17 is a partial isometric view of the step of filling the eye dropper from a bottle of liquid fragrance.

FIG. 18 is partial isometric view of the step of placing the liquid fragrance into the absorbent pad exposure openings of the chamber.

FIG. 19 is partial isometric view of the step of wiping excess liquid fragrance from the chamber.

FIG. 20 is partial isometric view of the step of placing an earring post through a person's ear lobe.

FIG. 21 is partial isometric view of the step of placing the fragrance chamber on the post extending beyond the person's ear lobe.

FIG. 22 is partial isometric view of the step of securing the fragrance chamber with a clasp on the earring post.

### BEST MODE FOR CARRYING OUT THE INVENTION

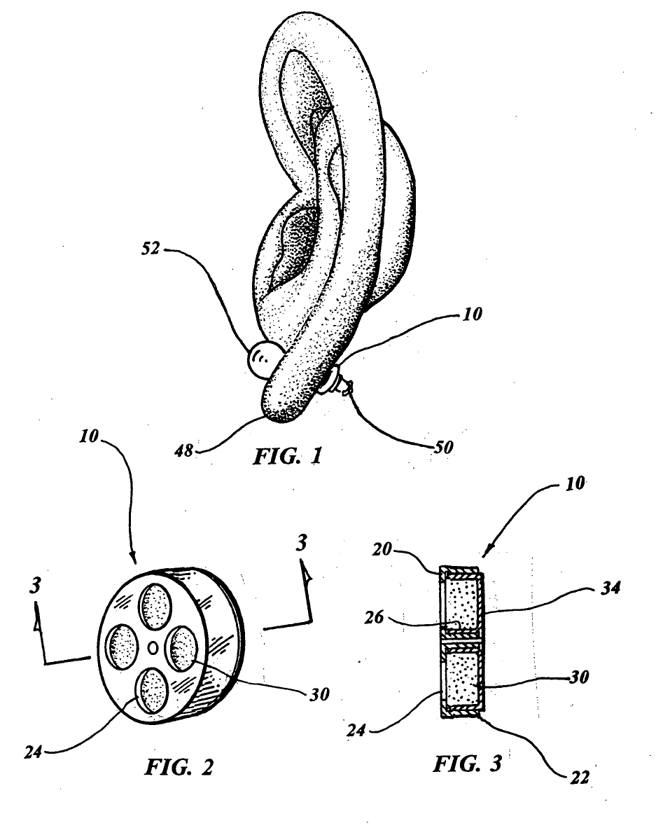

The best mode for carrying out the invention is presented in terms of a preferred embodiment for a disposable fragrance chamber 10, which is used to dispense a fragrance from behind a pierced ear lobe mounted on a pierced earring post 40 that is secured by an earring clasp 50 to which is attached an earring 52. The preferred embodiment of the disposable fragrance chamber 10, as shown in FIGS. 1 through 22, is comprised of a flat dish-shaped outer casing body 20 that incorporates an outward-extended perimeter edge 22, as shown best in FIGS. 3, 8-11 and 16.

The outer casing body 20 has a plurality of pad exposure openings 24 that are arranged in a symmetrical array, and an earring post column 26 with a bore 28 therethrough in the center. The earring post column's 26 height is identical to the perimeter edge's 22 height, and the outer casing body 20 preferably has a diameter from 10 to 12 mm, with the perimeter edge 22 having a height from 2 mm to 4 mm and a bore 28 from 0.7 to 0.9 mm within the earring post column 26. The pad exposure openings 24 preferably have a diameter from 3 mm to 4 mm, with the casing body wall thickness from 0.6 mm to 0.7 mm.

Preferably, the outer casing body 20, as shown alone in FIGS. 8-11, is formed of a transparent resilient thermoplastic such as cellulose acetate, chlorinated polyether, ethylene vinyl acetate, nylon, polycarbonate, polyethylene, polypropylene, vinyl or polyester.

An absorbent pad 30, having a central thru-hole 32, is disposed inside the outer casing body 20 within the perimeter edge 22, with the earring post column 26 penetrating the thru-hole 32, as shown in FIGS. 2, 3 and 12-16. The absorbent pad 30 preferably has an outside diameter from 8.8 mm to 10.6 mm, a height from 1.4 mm to 3.3 mm and a thru-hole 32 diameter from 3 mm to 4 mm.

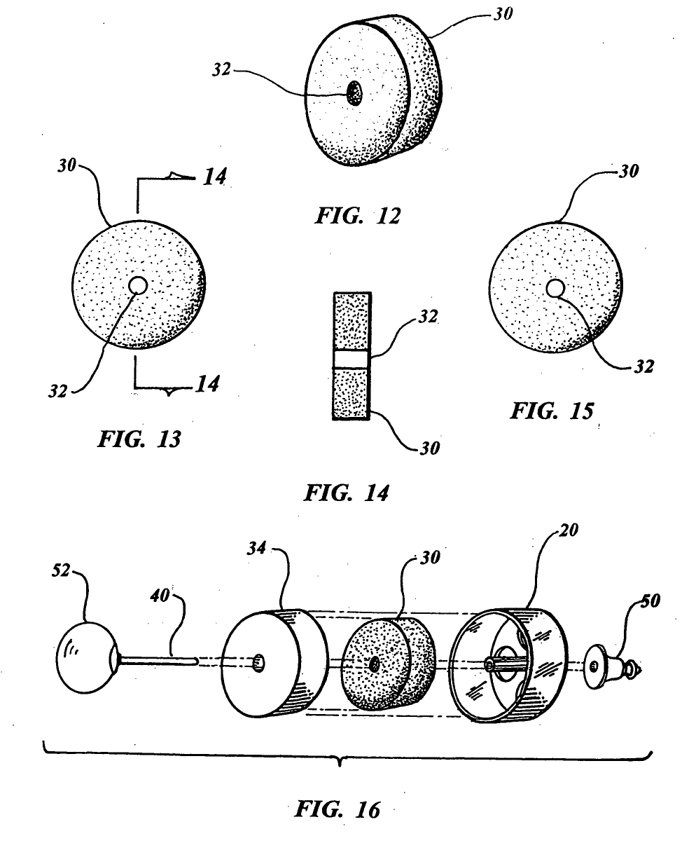

The absorbent pad 30 utilizes cotton felt as a preferred material, however other types of absorbent substances, such as open cell sponge or the like, may be used with equal ease. In order to be as concealed and non-obtrusive as possible, the absorbent pad 30 may be made using a flesh colored material. The absorbent pad 30, as shown completely removed from the invention in FIGS. 12-14, is made with conventional die-cut tooling.

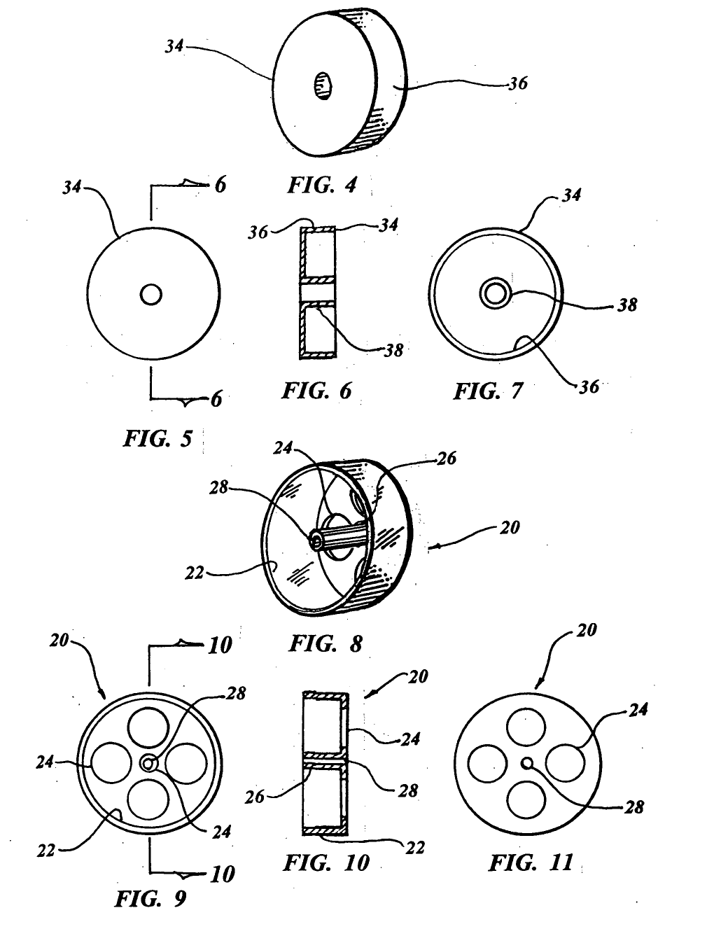

A flat, dish-shape inner casing lid 34, as shown in FIGS. 4-7, includes an extended perimeter lip 36 and an earring post column sleeve 38. The inner casing lid 34 is positioned inside the outer casing body 20 and contiguously engages its perimeter edge 22 of the outer casing body 20, with the lid perimeter lip 36. The earring post column 26 also interfaces with on the column sleeve 38, thereby frictionally maintaining the lid 34 on the body 20. The disposable fragrance chamber 10 is designed to allow an aromatic liquid to be introduced into the absorbent pad 30 through the pad exposure openings 24. Once the disposable fragrance chamber 10 is installed on a conventional earring post 40 between a person's ear lobe 48 and a conventional earring clasp 50, and after the pad 30 has been moistened with the aromatic liquid, a fragrance will then be released from the liquid into the surrounding environment.

The earring post column sleeve's 38 height is identical to the perimeter lip 36 height such that the sleeve 38 engages the outer casing body 20 when assembled together. The inner casing lid preferably has a diameter from 8.8 to 10.6 mm, with the perimeter lip having a height from 1.4 mm to 3.3 mm, and the earring post sleeve 38 having a diameter from 3 to 4 mm.

The inner casing lid 34 in the preferred embodiment is made of a translucent resilient thermoplastic. The thermoplastic may be cellulose acetate, chlorinated polyether, ethylene vinyl acetate, nylon, polycarbonate, polyethylene, polypropylene, vinyl or polyester.

The fragrance chamber outer casing body 20 and inner casing lid 34 mate together with press fit or with sufficient resistance to remain attached together when assembled. The absorbent pad thru-hole 32 is sized to interface intimately with the inner casing lid 34 and the earring post column sleeve 38, while allowing sufficient clearance for the pad 30.

While any aromatic liquid introduced into the absorbent pad 30 through the pad exposure openings 24 may be used, the preferred aromatic liquids are perfumes, essential oils, and insect repellents.

During use, the process of dispensing a fragrance from the assembled chamber 10 consists of the following steps:

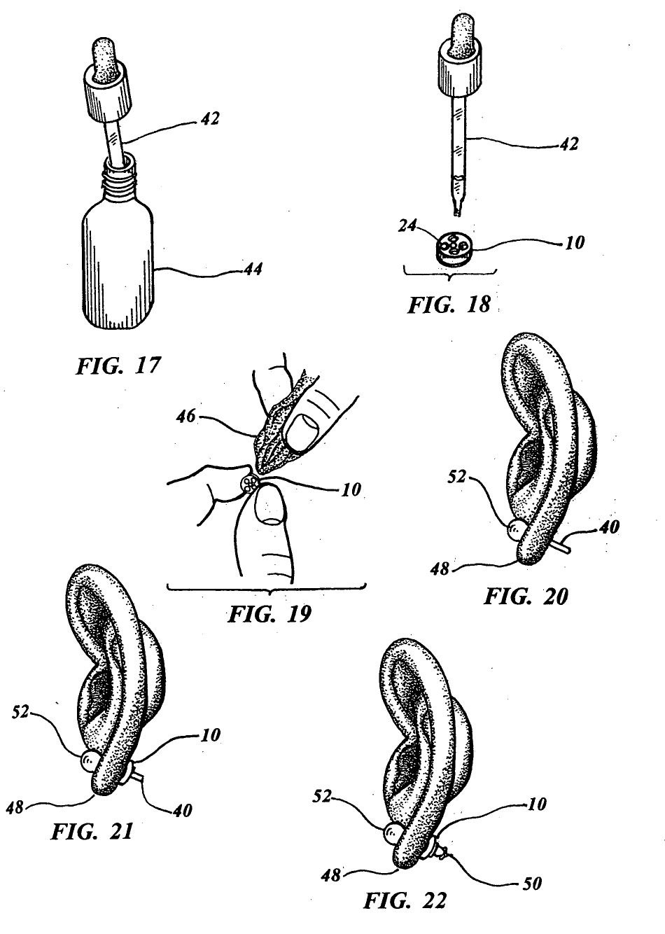

a) filling a dropper 42, such as a conventional eye dropper, with an aromatic liquid from a container 44, as shown in FIG. 17,

b) applying the aromatic liquid from the dropper 42 into the absorbent pad 30 through a set of pad exposure openings 24 located on the chamber 10, as shown in FIG. 18,

c) wiping excess liquid if any, from the chamber with a disposable tissue 46, or the like, as shown in FIG. 19,

d) placing the earring post 40 through a person's ear lobe 48, as shown in FIG. 20,

e) placing the chamber 10 on the earring post 40 that extends beyond the ear lobe 48, as shown in FIG. 21, and

f) securing the chamber 10 with an earring clasp 50, as shown in FIG. 22, thereby permitting a fragrance to be emitted from the aromatic liquid into a surrounding environment.

While the invention has been described in detail and pictorially shown in the accompanying drawings, it is not to be limited to such details, since many changes and modifications may be made to the invention without departing from the spirit and scope thereof. Hence, it is described to cover any and all modifications and forms which may come within the language and scope of the appended claims.

1. A disposable fragrance chamber for dispensing a fragrance, wherein said disposable fragrance chamber comprises an enclosing casing body having pad exposure openings and a lid with an absorbent pad therebetween, thereby permitting an aromatic liquid to be applied into the absorbent pad through the pad exposure openings and emit a fragrance from the aromatic liquid into the surrounding environment when the fragrance chamber is installed on an earring post between a person's ear lobe and a clasp.

2. A disposable fragrance chamber for dispensing a fragrance that is used in combination with an earring having a post for penetrating a person's ear lobe and a clasp wherein said fragrance chamber comprises:
a) a flat, dish-shaped outer casing body incorporating an outward-extended perimeter edge, said outer casing body having a plurality of pad exposure openings, and an earring post column having a earring post bore therethrough,
b) an absorbent pad having a central thru-hole disposed inside the outer casing body within the perimeter edge with the earring post column penetrating the thru-hole, and
c) a flat, dish-shape inner casing lid having an extended perimeter lip and a earring post column sleeve with the inner casing lid positioned inside the outer casing body contiguously engaging the perimeter edge and perimeter lip, as well as the earring post on the column sleeve, thereby permitting an aromatic liquid to be applied into the absorbent pad through the pad exposure openings, and to emit a fragrance from the aromatic liquid into surrounding environment when the fragrance chamber is installed on the earring post between the person's ear lobe and the clasp.

3. The disposable fragrance chamber as recited in claim 2 wherein said earring post column with a bore therethrough and the earring post column sleeve are centrally located and aligned such that the earring post column sleeve slides over the earring post column with a slip fit when the outer casing body and inner casing lid are assembled together.

4. The disposable fragrance chamber as recited in claim 2 wherein said outer casing body earring post column's height is identical to the perimeter edge height.

5. The disposable fragrance chamber as recited in claim 2 wherein said outer casing body further comprises a diameter from 10 to 12 mm, with the perimeter edge having a height from 2 mm to 4 mm and a bore from 0.7 to 0.9 mm.

6. The disposable fragrance chamber as recited in claim 2 wherein said outer casing body pad exposure openings have a diameter from 3 mm to 4 mm and said outer casing having a wall thickness from 0.6 mm to 0.7 mm.

7. The disposable fragrance chamber as recited in claim 2 wherein said outer casing body further comprises a transparent resilient thermoplastic.

8. The disposable fragrance chamber as recited in claim 7 wherein said thermoplastic is selected from the group consisting of cellulose acetate, chlorinated polyether, ethylene vinyl acetate, nylon, polycarbonate, polyethylene, polypropylene, vinyl and polyester.

9. The disposable fragrance chamber as recited in claim 2 wherein said absorbent pad thru-hole interfaces intimately with said inner casing lid earring post sleeve.

10. The disposable fragrance chamber as recited in claim 2 wherein said absorbent pad further having an outside diameter from 8.8 mm to 10.6 mm, a height from 1.4 mm to 3.3 mm and a thru-hole diameter from 3 mm to 4 mm.

11. The disposable fragrance chamber as recited in claim 2 wherein said absorbent pad further comprises cotton felt.

12. The disposable fragrance chamber as recited in claim 2 wherein said absorbent pad further comprises a flesh color material.

13. The disposable fragrance chamber as recited in claim 2 wherein said inner casing lid's earring post sleeve's height is identical to the perimeter lip height.

14. The disposable fragrance chamber as recited in claim 2 wherein said inner casing lid further having a diameter from 8.8 to 10.6 mm, with the perimeter lip having a height from 1.4 mm to 3.3 mm.

15. The disposable fragrance chamber as recited in claim 2 wherein said inner casing lid earring post sleeve further comprises a diameter from 3 to 4 mm.

16. The disposable fragrance chamber as recited in claim 2 wherein said inner casing lid further comprises a translucent resilient thermoplastic.

17. The disposable fragrance chamber as recited in claim 16 wherein said thermoplastic is selected from the group consisting of cellulose acetate, chlorinated polyether, ethylene vinyl acetate, nylon, polycarbonate, polyethylene, polypropylene, vinyl and polyester.

18. The disposable fragrance chamber as recited in claim 2 further comprising said outer casing body and said inner casing lid mate together with press fit.

19. The disposable fragrance chamber as recited in claim 2 wherein said liquid to be introduced into the absorbent pad through the pad exposure openings is selected from the group consisting of perfumes, essential oils, and insect repellents.

20. A process of dispensing a fragrance from a chamber held in place on a pierced earring comprising the steps of:
a) filling a dropper with a liquid from a container,
b) applying the aromatic liquid into an absorbent pad through a set of pad exposure openings located on said chamber,
c) wiping excess liquid from said chamber,
d) placing an earring post through a person's ear lobe,
e) placing said chamber on the post extending beyond the ear lobe, and
f) securing said chamber with an earring clasp, thereby permitting a fragrance to be emitted from the aromatic liquid into a surrounding environment.
